(19) United States
(12) Patent Application Publication
(10) Pub. No.: US 2004/0180029 A1
(43) Pub. Date: Sep. 16, 2004

(54) Cosmetic compositions comprising at least one alkylamphohydroxyalkylsulphonate amphoteric surfactant and at least one ceramide, and uses thereof

(76) Inventor: Mireille Maubru, Chatou (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/739,051

(22) Filed: Dec. 19, 2003

Related U.S. Application Data
(60) Provisional application No. 60/439,465, filed on Jan. 13, 2003.

(30) Foreign Application Priority Data
Dec. 19, 2002 (FR) 02 16191

(51) Int. Cl. A61K 7/075; A61K 7/08

(52) U.S. Cl. 424/70.21

(57) ABSTRACT

The present disclosure relates to novel cosmetic compositions comprising, in a cosmetically acceptable medium, at least one amphoteric surfactant chosen from alkylamphohydroxyalkylsulphonates and at least one ceramide compound.

The present disclosure further relates to cosmetic compositions that impart improved cosmetic properties to keratin fibers, such as improved disentangling, smoothing, volume, lightness, softness, flexibility, and manageability.

This application claims benefit of U.S. Provisional Application No. 60/439,465, filed Jan. 13, 2003.

The present disclosure relates to novel cosmetic compositions comprising, in a cosmetically acceptable medium, at least one amphoteric surfactant chosen from alkylamphohydroxyalkylsulphonates, and at least one ceramide compound.

It is known that hair that has been “sensitized” (i.e., damaged and/or embrittled) due to varying degrees of exposure to atmospheric agents, as well as mechanical and/or chemical treatments, such as dyeing, bleaching and/or permanent-waving, is often difficult to disentangle and to style, and can lack softness. Without being bound by theory, it is believed that the reason for this is that, due to the action of the attacking factors (e.g., atmospheric agents, mechanical and/or chemical treatments), the hair loses some of its constituents such as ceramides and proteins.

Ceramides and the analogues thereof are known to protect and/or repair the skin and/or hair fibers against attack from the various agents and treatments mentioned above. For example, they can have a barrier effect that limits the loss of proteins, and they can also reinforce the cohesion of the cuticle.

Since it is believed that the protection and/or care afforded by ceramides can be proportionately higher when there is a larger amount present on the hair or the skin, the present inventor sought to improve the fixing and/or deposition of compounds of ceramide type onto and/or into the hair or onto and/or into the skin, as well as to improve the cosmetic properties of compositions comprising at least one ceramide.

Thus, one aspect of the present disclosure relates to cosmetic compositions, such as detergents, which have improved cosmetic properties, for example, relating to disentangling, smoothing, flexibility, manageability and softness of keratin fibers, such as hair. Another aspect of the present disclosure relates to improving the fixing and/or deposition of compounds of ceramide type onto and/or into keratin materials such as the hair and/or the skin.

The present inventor has found that at least one alkylamphohydroxyalkylsulphonate amphoteric surfactant combined with at least one ceramide compound will allow at least one of the benefits described above to be achieved.

Alkylamphohydroxyalkylsulphonate amphoteric surfactants have been recommended in detergent cosmetic compositions. They have been described, for example, in Patent Application No. WO 99/36054. However, hairwashing compositions using these surfactants alone may not always result in satisfactory cosmetic properties.

Thus, one aspect of the present disclosure is a cosmetic composition comprising, in a cosmetically acceptable medium, at least one amphoteric surfactant chosen from alkylamphohydroxyalkylsulphonates and the salts thereof, and at least one ceramide compound.

Another aspect of the present disclosure is the use of at least one amphoteric surfactant chosen from alkylamphohydroxyalkylsulphonates and the salts thereof to improve the fixing and/or deposition of ceramide compounds onto and/or into keratin materials, such as the hair and/or the skin.

Yet another aspect of the present disclosure is the use of a composition as disclosed herein to improve at least one property of the hair chosen from disentangling, smoothing, volume, lightness, softness, flexibility, and manageability.

Still another aspect of the present disclosure is the use of at least one amphoteric surfactant chosen from alkylamphohydroxyalkylsulphonates and the salts thereof in a cosmetic composition comprising at least one ceramide compound, to improve at least one property of the hair chosen from disentangling, smoothing, volume, lightness, softness, flexibility and manageability.

Still yet another aspect of the present disclosure relates to a method for treating keratin materials, such as the hair, comprising the application of cosmetic compositions as-disclosed herein to keratin materials.

As disclosed herein, the term “keratin materials” means hair, eyelashes, eyebrows, skin, nails, mucous membranes, and the scalp. In one embodiment, the keratin material used herein is hair.

As disclosed herein, the term “keratin fibers” means hair, eyelashes and eyebrows. In one embodiment, the keratin fibers used herein are hair.

The various aspects of the disclosure are described in greater detail below. All the meanings and definitions of the compounds as disclosed herein, are valid for all aspects of the disclosure.

According to the present disclosure, the expression “ceramide compound” means a compound chosen from natural and synthetic ceramides, glycoceramides, pseudoceramides, and neoceramides.

Ceramide compounds are described, for example, in Patent Application Nos. DE 4,424,530, DE 4,424,533, DE 4,402,929, DE 4,420,736, WO 95/23807, EP-A-0,646, 572, WO 95/16665, FR-2,673,179, EP-A-0,227,994, WO 94/07844, WO 94/24097 and WO 94/10131, the teachings of which are incorporated herein by reference.

Among the ceramide compounds that may be used according to the present disclosure, non-limiting mention may be made, for example, of compounds of formula (I) below:

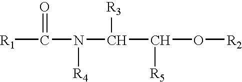

wherein:

R₁ is chosen from:

saturated and unsaturated, linear and branched C₁-C₅₀, for example, C₅-C₅₀, hydrocarbon-based radicals, wherein the alkyl radical may optionally be substituted with at least one hydroxyl group that may be esterified with an acid R₇COOH, wherein R₇ is chosen from saturated and unsaturated, linear and branched, C₁-C₃₅ hydrocarbon-based radicals, wherein the C₁-C₃₅ hydrocarbon-based radical may optionally be substituted with at least one hydroxyl group and wherein at least one hydroxyl of the radical R₇ may optionally be esterified with a fatty acid chosen from saturated and unsaturated, linear and branched, C₁-C₃₅ fatty acids that may optionally be substituted with at least one hydroxyl group,

radicals of the formula: R″—(NR°—CO)—R′, wherein R° is chosen from hydrogen and C₁-C₂₀ hydrocarbon-based radicals substituted with at least one hydroxyl group, and R′ and R″ are chosen from C₉ to C₃₀ hydrocarbon-based radicals, wherein R′ is a divalent radical, and

radicals of formula R₈—O—CO—(CH₂)ₚ, wherein R₈ is chosen from C₁-C₂₀ hydrocarbon-based radicals, and p is an integer ranging from 1 to 12;

R₂ is chosen from hydrogen, saccharide radicals, such as (glycosyl)ₙ, (galactosyl)ₘ and sulphogalactosyl radicals, sulphate residues, phosphate residues, a phosphorylethylamine radical, and a phosphorylethylammonium radical, wherein n is an integer ranging from 1 to 4, and m is an integer ranging from 1 to 8;

R₃ is chosen from hydrogen, and saturated and unsaturated C₁-C₃₃ hydrocarbon-based radicals that may be optionally substituted with at least one hydroxyl group, wherein the at least one hydroxyl group may optionally be esterified with an acid chosen from mineral acids (such as phosphoric acid for example) and R₇COOH acids, wherein R₇ is chosen from saturated and unsaturated, linear and branched, C₁-C₃₅ hydrocarbon-based radicals that may optionally be substituted with at least one hydroxyl group, and wherein the at least one hydroxyl of the radical R₇ may optionally be esterified with a fatty acid chosen from saturated and unsaturated, linear and branched, C₁-C₃₅ fatty acids that may optionally be substituted with at least one hydroxyl group, and further wherein at least one hydroxyl group may optionally be etherified with radicals chosen from (glycosyl)ₙ, (galactosyl)ₘ, sulphogalactosyl, phosphorylethylamine and phosphorylethylammonium radicals, wherein n is an integer ranging from 1 to 4, and m is an integer ranging from 1 to 8, and wherein R₃ may be optionally substituted with at least one C₁-C₁₄ alkyl radical; for example, R₃ may be chosen from C₁₅-C₂₆ α-hydroxyalkyl radicals, wherein the hydroxyl group may optionally be esterified with a C₁₆-C₃₀ α-hydroxy acid;

R4 is chosen from hydrogen, a methyl radical, an ethyl radical, saturated and unsaturated, linear and branched, C₃-C₅₀ hydrocarbon-based radicals that may optionally be substituted with at least one hydroxyl group, and radicals of formula —CH₂—CHOH—CH₂—O—R₆, wherein R₆ is chosen from C₁₀-C₂₆ hydrocarbon-based radicals and radicals of formula R₈—O—CO—(CH₂)ₚ, wherein R₈ is chosen from C₁-C₂₀ hydrocarbon-based radicals, and p is an integer ranging from 1 to 12,

R₅ is chosen from hydrogen, and saturated and unsaturated, linear and branched, C₁-C₃₀ hydrocarbon-based radicals that may optionally be substituted with at least one hydroxyl group, wherein the at least one hydroxyl may optionally be etherified with a radical chosen from (glycosyl)ₙ, (galactosyl)ₘ, sulphogalactosyl, phosphorylethylamine and phosphorylethylammonium radicals,

with the proviso that when R₃ and R₅ are both hydrogen, or when R₃ is a hydrogen and R₅ is a methyl, then R₄ is not chosen from hydrogen, methyl, and ethyl radicals.

Among the compounds of formula (I) above, non-limiting mention may be made of the ceramides and/or glycoceramides described by Downing in Journal of Lipid Research, Vol. 35, page 2060-2068,1994, and those described in French Patent Application FR-2,673,179, and the teachings of which are incorporated herein by way of reference.

Further among the ceramide compounds as disclosed herein, non-limiting mention may be made of the compounds of formula (I), wherein R₁ is chosen from saturated and unsaturated, optionally hydroxylated alkyl groups derived from C₁₄-C₂₂ fatty acids; R₂ is a hydrogen atom; and R₃ is chosen from linear C₁₁-C₁₇ radicals, such as C₁₃-C₁₅ radicals, which may optionally be substituted with at least one hydroxyl group.

Thus, in one aspect of the present disclosure, the at least one ceramide compound may be chosen from:

2-N-linoleoylaminooctadecane-1,3-diol,

2-N-oleoylaminooctadecane-1,3-diol,

2-N-palmitoylaminooctadecane-1,3-diol,

2-N-stearoylaminooctadecane-1,3-diol,

2-N-behenoylaminooctadecane-1,3-diol,

2-N-[2-hydroxypalmitoyl]aminooctadecane-1,3-diol,

2-N-stearoylaminooctadecane-1,3,4-triol, such as N-stearoylphytosphingosine, and

2-N-palmitoylaminohexadecane-1,3-diol.

The ceramide compounds of formula (I), wherein R₁ is chosen from saturated and unsaturated C₁₂-C₂₂ alkyl radicals derived from fatty acids; R₂ is chosen from galactosyl and sulphogalactosyl radicals; and R₃ is chosen from saturated and unsaturated C₁₂-C₂₂ hydrocarbon-based radicals, such as a CH(OH)—CH═CH—(CH₂)₁₂—CH₃ group, may also be used as disclosed herein.

By way of non-limiting example, mention may be made of the product comprising a mixture of glycoceramides, sold under the trade name GLYCOCER by the company Waitaki International Biosciences.

The compounds of formula (I) described in patent applications Nos. EP-A-0 227 994 and WO 94/07844 may also be used. Such compounds include, for example QUESTAMIDE H (bis(N-hydroxyethyl-N-cetyl)malonamide) sold by the company Quest.

N-Docosanoyl-N-methyl-D-glucamine, described in Patent Application WO 94/24097 may also be used.

The at least one ceramide compound may be present in the composition in an amount ranging from 0.0001% to 20% by weight, such as from 0.001% to 10% by weight, and for example, from 0.005% to 3% by weight, relative to the total weight of the composition.

The amphoteric surfactants chosen from alkylamphohydroxyalkylsulphonates may be of formula (II):

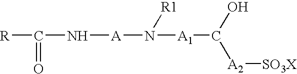

wherein:

R is chosen from saturated and unsaturated, linear and branched hydrocarbon-based radicals comprising from 5 to 29 carbon atoms,

For example, R may be chosen from mono- and polyunsaturated alkyl and alkenyl radicals comprising from 5 to 29 carbon atoms, such as from 7 to 22 carbon atoms, and for instance, from 9 to 17 carbon atoms,

R1 may be chosen from C₁-C₄ hydroxyalkyl radicals, such as hydroxyethyl,

A, A₁ and A₂, which may be identical or different, may be chosen from linear and branched C₁-C₁₀, for example, C₁-C₃ divalent alkylene radicals,

X is chosen from hydrogen, and mineral and organic cations such as:

that of an alkali metal (for example Na⁺ and K⁺), that of an alkaline-earth metal (Mg²⁺ and Ca²⁺), an NH₄⁺ ion, and ammonium ions derived from basic amino acids and/or from amino alcohols.

For example, according to one aspect of the present disclosure, the at least one amphoteric surfactant is chosen from alkylamphohydroxyalkylsulphonates of formula (II) wherein R is chosen from saturated, linear and branched alkyl radicals comprising from 7 to 29 carbon atoms, such as from 7 to 22 carbon atoms.

When X is an ammonium ion derived from an alkanolamine, the alkanolamine may be chosen from monoethanolamine, diethanolamine, triethanolamine and 3-amino-1,2-propanediol. When X is an ammonium ion derived from an amine, the amine may be chosen from basic amino acids, such as lysine, arginine, sarcosine, ornithine and citrulline.

For example, A may be the same as A₂ and both may be —CH₂CH₂—.

For further example, A₁ may be —CH₂—.

For another example, X may be Na⁺.

Among the surfactants of formula (II), non-limiting mention may be made of: cocoyl amphohydroxypropyl sulphonate salts, for example, the sodium salt, such as the product sold under the name MIRANOL CSE by the company Rhodia Chimie, and palmitoyl amphohydroxypropyl sulphonate salts.

As disclosed herein, the at least one amphoteric surfactant chosen from alkylamphohydroxyalkylsulphonates and the salts thereof may be present in the composition in an amount ranging from 0.1% to 30% by weight, such as from 1% to 20% by weight, for example, from 1.5% to 15% by weight, and for further example, from 2% to 8% by weight, relative to the total weight of the final composition.

The compositions as disclosed herein may also further comprise at least one additional surfactant, which may be present in the composition in an amount ranging from 0.1% to 60%, by weight such as, ranging from 3% to 40%, by weight, for example, ranging from 5% to 30%, relative to the total weight of the composition.

The at least one additional surfactant may be chosen from anionic, amphoteric, non-ionic and cationic surfactants, provided that the at least one additional surfactant is different from the at least one amphoteric surfactant chosen from alkylamphohydroxyalkylsulphonates and salts thereof.

Among the examples of the at least one additional surfactant as disclosed herein, non-limiting mention may be made of the following:

(i) Anionic Surfactant(s):

In the context of the present disclosure, their nature is not an important feature. Thus, as non-limiting examples of anionic surfactants that may be used alone and as mixtures, as disclosed herein, mention may be made of: salts (such as alkaline salts, for example sodium salts, ammonium salts, amine salts, amino alcohol salts and magnesium salts) of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates, alkyl sulphosuccinates, alkyl ether sulphosuccinates, alkylamide sulphosuccinates, alkyl sulphosuccinamates, alkyl sulphoacetates, alkyl ether phosphates, acyl sarcosinates, acyl isethionates, and N-acyltaurates, wherein the alkyl and acyl radicals of the above described compounds may comprise from 8 to 24 carbon atoms, and wherein the aryl radicals of the above described compounds may chosen from phenyl and benzyl groups. Among the anionic surfactants that can also be used, non-limiting mention may also be made of fatty acid salts, such as the salts of oleic, ricinoleic, palmitic and stearic acids, coconut oil acid and hydrogenated coconut oil acid; acyl lactylates wherein the acyl radical comprises from 8 to 20 carbon atoms. Weakly anionic surfactants can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, for example, those comprising from 2 to 50 ethylene oxide groups, and mixtures thereof.

For instance, as disclosed herein, an anionic surfactant may be chosen from sodium, magnesium and ammonium (C₁₂-C₁₄)alkyl sulphates; sodium, magnesium and ammonium (C₁₂-C₁₄)alkyl ether sulphates oxyethylenated with 2.2 mol of ethylene oxide; and sodium cocoyl isethionate.

For another instance, as disclosed herein, an anionic surfactant, may be chosen from alkyl sulphate salts and alkyl ether sulphate salts.

(ii) Nonionic Surfactant(s):

The non-ionic surfactants are, themselves also, compounds that are well known per se (see, for example, “Handbook of Surfactants” by M. R. Porter, published by Blackie & Son (Glasgow and London), 1991, pp. 116-178) and, in the context of the present disclosure, their nature is not important. Thus, non-limiting examples of non-ionic surfactants as disclosed herein, include: polyethoxylated, polypropoxylated and polyglycerolated fatty acids, alkylphenols, α-diols and alcohols having a fatty chain comprising, for example, 8 to 18 carbon atoms, it being possible for the number of ethylene oxide and/or propylene oxide groups to range, for example, from 2 to 50, and for the number of glycerol groups to range, for example, from 2 to 30. Non-limiting mention may also be made of copolymers of ethylene oxide and of propylene oxide, condensates of ethylene oxide and of propylene oxide with fatty alcohols; polyethoxylated fatty amides comprising, for example, from 2 to 30 mol of ethylene oxide, polyglycerolated fatty amides comprising, for instance, from 1 to 5, such as 1.5 to 4, glycerol groups; oxyethylenated fatty acid esters of sorbitan comprising from 2 to 30 mol of ethylene oxide; fatty acid esters of sucrose; fatty acid esters of polyethylene glycol; alkylpolyglycosides; N-alkylglucamine derivatives; amine oxides, such as (C₁₀-C₁₄)alkylamine oxides and N-acylaminopropylmorpholine oxides. For instance, as disclosed herein, the non-ionic surfactants may be chosen from alkylpolyglycosides.

(iii) Amphoteric Surfactant(s):

Non-limiting examples of the at least one additional amphoteric surfactant, include those chosen from: aliphatic secondary and tertiary amine derivatives wherein the aliphatic radical is chosen from linear and branched chains comprising from 8 to 22 carbon atoms and further comprising at least one water-soluble anionic group (for example carboxylate, sulphonate, sulphate, phosphate and phosphonate). Non-limiting mention may also be made of (C₈-C₂₀)alkylbetaines, sulphobetaines, (C₈-C₂₀)alkylamido(C₁-C₆)alkyl betaines and (C₈-C₂₀)alkylamido(C₁-C₆)alkylsulphobetaines.

For example, among the amine derivatives, non-limiting mention may be made of the products sold under the name MIRANOL, as described in U.S. Pat. Nos. 2,528,378 and 2,781,354 and having the structures:

R₂—CONHCH₂CH₂—N(R₃)(R₄)(CH₂COO—) (III)

wherein:

R₂ is chosen from alkyl radicals derived from the acid R₂—COOH present in hydrolysed coconut oil, and from heptyl, nonyl and undecyl radicals,

R₃ is a β-hydroxyethyl group, and

R₄ is a carboxymethyl group;

and

R₅-QONHCH₂CH₂—N(B)(D) (IV)

wherein:

B is chosen from —CH₂CH₂OX′ groups,

D is chosen from —(CH₂)_{z}—Y′ groups, wherein z is an integer chosen from 1 and 2, X′ is chosen from —CH₂CH₂—COOH groups and hydrogen, Y′ is chosen from —COOH and the —CH₂—CHOH—SO₃H radical, R₅ is chosen from alkyl radicals of an acid R₉—COOH found in coconut oil and in hydrolysed linseed oil, alkyl radicals, such as C₇, C₉, C₁₁ and C₁₃ alkyl radicals, a C₁₇ alkyl radical and its iso form, and an unsaturated C₁₇ radical and R₉ is chosen from saturated and unsaturated, linear and branched alkyl groups having from 8 to 22 carbon atoms, such as from 8 to 16 carbon atoms.

These compounds are classified in the CTFA dictionary, 5^{th} edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylampho-dipropionate, disodium capryloamphodipropionate, lauroamphodipropionic acid, and cocoamphodipropionic acid.

By way of non-limiting example, mention may be made of the cocoamphodiacetate sold under the trade name MIRANOL C2M concentrate by the company Rhodia Chimie.

In the compositions as disclosed herein, mixtures of the additional surfactants can be used, for instance, mixtures of anionic surfactants, and mixtures of anionic surfactants with amphoteric and/or nonionic surfactants. For instance, in the compositions as disclosed herein, a mixture of surfactants may comprise at least one anionic surfactant and at least one amphoteric surfactant.

Cationic surfactants may also be used, among non-limiting examples of which, mention may be made of: optionally polyoxyalkylenated primary, secondary and tertiary fatty amine salts; quaternary ammonium salts, such as tetraalkylammonium, alkylamidoalkyltrialkylammonium, trialkylbenzylammonium, trialkylhydroxyalkylammonium and alkylpyridinium chlorides and/or bromides; imidazoline derivatives; and amine oxides of cationic nature.

Additionally, the compositions as disclosed herein may further comprise viscosity regulators such as electrolytes or thickeners. Non-limiting mention may be made, for example, of sodium chloride; sodium xylenesulphonate; scleroglucans; xanthan gums; fatty acid alkanolamides; alkyl ether carboxylic acid alkanolamides optionally oxyethylenated with up to 5 mol of ethylene oxide, such as the product sold under the name “AMINOL A15” by the company Chem Y; crosslinked polyacrylic acids and acrylic acid/C₁₀-C₃₀ alkyl acrylate crosslinked copolymers. The viscosity regulators may be used in the compositions as disclosed herein in amounts that may be up to 10% by weight, relative to the total weight of the composition.

The compositions as disclosed herein, may also comprise up to 5% by weight of additives chosen from nacreous agents and opacifiers that are known in the art, for instance additives chosen from coated and uncoated titanium oxides, sodium and magnesium palmitate, sodium and magnesium stearate and hydroxystearate, fatty-chain acyl derivatives such as ethylene glycol and polyethylene glycol monostearates and distearates, fatty-chain ethers (for example of C8-C30), for instance distearyl ether and 1-(hexadecyloxy)-2-octadecanol, cyclodextrins, and fatty alcohols such as cetyl alcohol, stearyl alcohol and behenyl alcohol.

The compositions as disclosed herein may also comprise at least one cationic polymer.

The cationic polymers that may be used in accordance with the present disclosure may be chosen from any of those already known per se as improving the cosmetic properties of hair treated with detergent compositions, for example, the cationic polymers described in patent application No. EP-A-0,337,354 and French Patent Applications FR-A-2 270 846, 2 383 660, 2 598 611, 2 470 596 and 2 519 863.

As used herein, the term “cationic polymer” means any polymer comprising cationic groups and/or groups that may be ionizable into cationic groups.

Among the cationic polymers that may be used as disclosed herein, non-limiting examples include quaternary cellulose ether derivatives, such as the products sold under the name “JR 400” by the company Amerchol; cyclopolymers, for example, the homopolymers of diallyldimethylammonium salt, the copolymers of diallyldimethylammonium salt, and of acrylamide, for instance, the chlorides, sold under the names “MERQUAT 100”, “MERQUAT 550” AND “MERQUAT S” by the company Ondeo, non-cellulose-based cationic polysaccharides such as guar gums modified with 2,3-epoxypropyltrimethylammonium chloride, for example sold under the name “JAGUAR C13S” by the company Rhodia Chimie.

In one aspect of the present disclosure, the at least one cationic polymer may be chosen from:

homopolymers of diallyldimethylammonium salt and copolymers of diallyldimethylammonium salt and of acrylamide,

quaternary cellulose ether derivatives,

non-cellulose-based cationic polysaccharides,

polymers comprising repeating units of the formula:

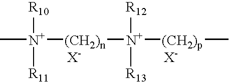

wherein R₁₀, R₁₁, R₁₂ and R₁₃, which may be identical or different, are chosen from C₁-C₄ alkyl and hydroxyalkyl radicals, n and p are integers ranging from 2 to 20, and X is an anion derived from a mineral or organic acid.

As disclosed herein, the at least one cationic polymer may be present in the composition in an amount ranging from 0.001% to 10% by weight, for example, from 0.005% to 5% by weight, and for instance, from 0.01% to 3% by weight, relative to the total weight of the composition.

The compositions as disclosed herein may comprise foam synergists such as C₁₀-C₁₈ 1,2-alkanediols, and fatty alkanolamides derived from monoethanolamine and diethanolamine.

The composition as disclosed herein may also comprise at least one additive chosen from fragrances; preserving agents; sunscreens; anionic, non-ionic and amphoteric polymers; proteins; protein hydrolyzates; linear and branched C₁₆-C₄₀ fatty acids, such as 18-methyleicosanoic acid; hydroxy acids; vitamins; panthenol; plant, animal, mineral and synthetic oils; volatile and non-volatile silicones; fatty esters and any other additive conventionally used in cosmetics that does not affect the properties of the compositions as disclosed herein.

These at least one additive may be present in the composition as disclosed herein in an amount ranging from 0% to 50% by weight, relative to the total weight of the composition. The precise amount of each additive can be readily determined by a person of ordinary skill in the art, depending on the nature and function of the at least one additive.

The cosmetically acceptable medium may comprise solely of water, or of a mixture of water and of at least one cosmetically acceptable solvent such as a C₁-C₄ lower alcohol, for instance ethanol, isopropanol, tert-butanol and n-butanol; alkylene glycols, for instance propylene glycol, and glycol ethers.

The composition may comprise, for example, from 50% to 95% by weight of water, relative to the total weight of the composition.

The compositions as disclosed herein may have a final pH ranging approximately from 3 to 10. For example, the pH may range approximately from 4 to 8. The pH may be adjusted to the desired value conventionally, by adding a base (such as organic and mineral bases) to the composition, for example aqueous ammonia, and primary, secondary and tertiary (poly)amines, for instance monoethanolamine, diethanolamine, triethanolamine, isopropanolamine and 1,3-propanediamine, or alternatively, by adding an acid, such as a carboxylic acid, for instance citric acid.

The compositions as disclosed herein may be used for washing and/or treating keratin materials such as the hair, the skin, the eyelashes, the eyebrows, the nails, the lips, and the scalp; for instance, the hair.

For example, the compositions as disclosed herein, may be detergent compositions such as shampoos, shower gels and bubble baths. When the compositions as disclosed herein are detergents, the compositions comprise a washing base, which is generally aqueous. The washing base comprises at least one additional surfactant.

The at least one additional surfactant may be chosen from, for example, the anionic and non-ionic surfactants as defined above.

The quantity and quality of the washing base are those that are sufficient to give the final composition satisfactory foaming power and/or detergent power.

These detergent compositions can be, for example, foaming compositions wherein the foaming power of the compositions as disclosed herein, such that the foam height, is approximately greater than 75 mm, for example, greater than 100 mm, measured according to the modified Ross-Miles method (NF T 73-404/ISO696). The modifications to the method are the following:

The measurement is performed at a temperature of 22° C. with osmosed water. The concentration of the solution is 2 g/L. The drop height is 1 m. The amount of composition dropped is 200 ml. The 200 ml of composition fall into a measuring cylinder with a diameter of 50 mm, containing 50 ml of the test composition. The measurement is performed 5 minutes after stopping the flow of the composition.

Thus, as disclosed herein, the washing base can be present in the composition in an amount ranging from 3% to 50% by weight, for example, from 6% to 35% by weight, and such as from 8% to 25% by weight, relative to the total weight of the composition.

Another aspect of the present disclosure is a method for treating keratin materials such as the skin and the hair, comprising the application to the keratin materials of a cosmetic composition asdisclosed herein, and then optionally rinsing with water.

Thus, the method as disclosed herein will allow the maintenance of the hair and the treatment, care, washing and/or the removal of makeup from the skin, the hair and any other keratin material.

The compositions as disclosed herein may also be in the form of compositions chosen from rinse-out conditioners, leave-in conditioners, permanent-waving, hair-relaxing, dyeing, bleaching, rinse-out compositions to be applied before and/or after dyeing, permanent-waving, relaxing the hair, and compositions to be applied between the two steps of permanent-waving and hair-relaxing operations.

The compositions as disclosed herein, may also be in the form of washing compositions for the skin, such as in the form of bath and shower solutions and gels, and makeup-removing products.

The compositions as disclosed herein may be in the form chosen from aqueous and aqueous-alcoholic lotions, gels, milks, creams, emulsions, thickened lotions and mousses, and the compositions may be used for the skin, the nails, the eyelashes, the lips and, for example, the hair.

Throughout the text hereinabove and hereinbelow, the percentages expressed are on a weight basis.

The disclosure will now be illustrated more fully with the aid of the examples that follow, which cannot be considered as limiting it to the embodiments described.

In the examples, AM means active material.

### EXAMPLE 1

A shampoo composition corresponding to the compositions disclosed herein was prepared:

| Composition | Example 1 | |
|---|---|---|
| Sodium lauryl ether sulphate (70/30 C12/C14) comprising | 15.5 | g AM |
| 2.2 mol of ethylene oxide | | |
| Sodium cocoylamphohydroxypropylsulphonate, as an | 2.2 | g AM |
| aqueous solution comprising 32% active material, sold | | |
| under the name MIRANOL CSE by the company Rhodia | | |
| Guar gum modified with | 0.1 | g |
| 2,3-epoxypropyltrimethylammonium chloride, | | |
| sold under the name JAGUAR | | |
| C13 S by the company Rhodia | | |
| 2-N-Oleoylaminooctadecane-1,3-diol | 0.01 | g |
| Polydimethylsiloxane of viscosity 300 000 cSt, sold under | 2.7 | g |
| the name DC 200 Fluid 300 000 by the company Dow | | |
| Corning | | |
| Distearyl ether | 1.5 | g |
| Behenyl alcohol | 1.5 | g |
| Coconut acid monoisopropanolamide | 1 | g |
| Crosslinked polyacrylic acid | 0.2 | g |
| Preserving agents | Qs | |
| Fragrance | Qs | |
| Citric acid qs | pH 7 | |
| Demineralized water qs | 100 | g |

A shampoo wash was performed by applying about 12 g of the composition to moistened hair. The shampoo was worked into a lather and then was rinsed out thoroughly with water.

The hair washed with these compositions disentangled easily, felt smooth from the root to the end, and was manageable (flexible).

What is claimed is:

1. A cosmetic composition, comprising, in a cosmetically acceptable medium, at least one amphoteric surfactant chosen from alkylamphohydroxyalkylsulphonates and salts thereof, and at least one ceramide compound.

2. The composition according to claim 1, wherein the at least one ceramide compound is chosen from compounds of formula (I): 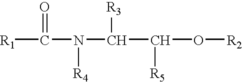
wherein:
is chosen from:
saturated and unsaturated, linear and branched C₁-C₅₀, hydrocarbon-based radicals, wherein the radical may optionally be substituted with at least one hydroxyl group that may optionally be esterified with an acid R₇COOH, wherein R₇ is chosen from saturated and unsaturated, linear and branched C₁-C₃₅ hydrocarbon-based radicals that may optionally be substituted with at least one hydroxyl group, wherein at least one hydroxyl of the radical R₇ may optionally be esterified with a fatty acid chosen from saturated and unsaturated, linear and branched C₁-C₃₅ fatty acids that may optionally be substituted with at least one hydroxyl group,
radicals of R″—(NR°—CO)—R′, wherein R° is chosen from hydrogen and C₁-C₂₀ hydrocarbon-based radicals comprising at least one hydroxyl group, R′ and R″ are chosen from C₉ to C₃₀ hydrocarbon-based radicals, and wherein R′ is a divalent radical, and
radicals of R₈—O—CO—(CH₂)ₚ, wherein R₈ is chosen from C₁-C₂₀ hydrocarbon-based radicals, and p is an integer ranging from 1 to 12;
R₂ is chosen from hydrogen, saccharide radicals, sulphate and phosphate residues, phosphorylethylamine radicals and phosphorylethylammonium radicals;
R₃ is chosen from hydrogen and saturated and unsaturated, C₁-C₃₃ hydrocarbon-based radicals that may optionally be substituted with at least one hydroxyl group, Wherein the at least one hydroxyl may optionally be esterified with an acid chosen from mineral acids and acid R₇COOH, wherein R₇ is chosen from saturated and unsaturated, linear and branched C₁-C₃₅ hydrocarbon-based radicals that may optionally be substituted with at least one hydroxyl group, wherein the at least one hydroxyl of the radical R₇ may optionally be esterified with a fatty acid chosen from saturated and unsaturated, linear and branched C₁-C₃₅ fatty acids that may optionally be substituted with at least one hydroxyl group, and wherein the at least one hydroxyl may optionally be etherified with a radical chosen from (glycosyl)ₙ, (galactosyl)ₘ, sulphogalactosyl, phosphorylethylamine and phosphorylethylammonium radicals, wherein n is an integer ranging from 1 to 4 and m is an integer ranging from 1 to 8, and further wherein R₃ may optionally be substituted with at least one C₁-C₁₄ alkyl radical;
R₄ is chosen from hydrogen, methyl and ethyl radicals, saturated and unsaturated, linear and branched C₃-C₅₀ hydrocarbon-based radicals that may optionally be substituted with at least one hydroxyl group, and —CH₂—CHOH—CH₂—O—R₆ radicals, wherein R₆ is chosen from C₁₀-C₂₆ hydrocarbon-based radicals and R₈—O—CO—(CH₂)ₚ radicals, wherein R₈ is chosen from C₁-C₂₀ hydrocarbon-based radicals, and p is an integer ranging from 1 to 12,
R₅ is chosen from hydrogen, and saturated and unsaturated, linear and branched C₁-C₃₀ hydrocarbon-based radicals that may optionally be substituted with at least one hydroxyl group, wherein the at least one hydroxyl may optionally be etherified with a radical chosen from (glycosyl)ₙ, (galactosyl)ₘ, sulphogalactosyl, phosphorylethylamine and phosphorylethylammonium radicals,
with the proviso that when R₃ and R₅ are both hydrogen, or when R₃ is hydrogen and R₅ is methyl, then R₄ is not chosen from hydrogen, methyl and ethyl radicals.

3. The composition according to claim 2, wherein R₁ is chosen from saturated and unsaturated, linear and branched hydrocarbon-based radicals, defined as in claim 2 and having from 5 to 50 carbon atoms.

4. The composition according to claim 2, wherein R₁ is chosen from radicals of R″—(NR°—CO)—R′, wherein R° is chosen from hydrogen and monohydroxylated, C₁-C₂₀ hydrocarbon-based radicals.

5. The composition according to claim 2, wherein the saccharide radicals are chosen from (glycosyl)ₙ, (galactosyl)ₘ and sulphogalactosyl radicals, wherein n is an integer ranging from 1 to 4 and m is an integer ranging from 1 to 8.

6. The composition according to claim 2, wherein R₃ is chosen from C₁₅-C₂₆ α-hydroxyalkyl radicals, wherein the hydroxyl group may optionally be esterified with a C₁₆-C₃₀ α-hydroxy acid.

7. The composition according to claim 1, wherein the at least one ceramide compound is chosen from:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol,
2-N-palmitoylaminohexadecane-1,3-diol,
bis(N-hydroxyethyl-N-cetyl)malonamide,
cetylic acid N-(2-hydroxyethyl)-N-(3-cetyloxy-2-hydroxypropyl)amide, and
N-docosanoyl-N-methyl-D-glucamine.

8. The composition according to claim 7, wherein the 2-N-stearoylaminooctadecane-1,3,4-triols are chosen from N-stearoylphytosphingosine.

9. The composition according to claim 1, wherein the at least one amphoteric surfactant is chosen from alkylamphohydroxyalkylsulphonate compounds of formula (II) and salts thereof: 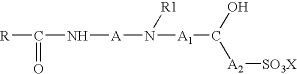
wherein:
R is chosen from saturated and unsaturated, linear and branched C₅-C₂₉ hydrocarbon-based radicals,
R₁ is chosen from C₁-C₄ hydroxyalkyl radicals,
A, A₁ and A₂, which may be identical or different, are chosen from linear and branched C₁-C₁₀ divalent alkylene radicals,
X is chosen from hydrogen, mineral cations, and organic cations.

10. The composition according to claim 9, wherein R1 is chosen from hydroxyethyl.

11. The composition according to claim 9, wherein A, A₁ and A₂, which may be identical or different, are chosen from linear and branched C₁-C₃ divalent alkylene radicals.

12. The composition according to claim 9, wherein R is chosen from mono- and polyunsaturated alkyl and alkenyl C₅-C₂₉ radicals.

13. The composition according to claim 12, wherein R is chosen from mono- and polyunsaturated alkyl and alkenyl C₇-C₂₂ radicals.

14. The composition according to claim 13, wherein R is chosen from mono- and polyunsaturated alkyl and alkenyl C₉-C₁₇ radicals.

15. The composition according to claim 9, wherein A and A₂ are both —CH₂CH₂—.

16. The composition according to claim 9, wherein A₁ is —CH₂—.

17. The composition according to claim 9, wherein the mineral and organic cations are chosen from alkali metals, alkaline-earth metals, an NH₄⁺ ion, and ammonium ions derived from basic amino acids and/or amino alcohols.

18. The composition according to claim 17, wherein the alkali metal cationic ions are chosen from Na⁺ and K⁺.

19. The composition according to claim 17, wherein the alkaline-earth metal cationic ions are chosen from Mg²⁺ and Ca²⁺.

20. The composition according to claim 1, wherein the at least one amphoteric surfactant is chosen from cocoyl amphohydroxypropylsulphonate salts and palm itoyl amphohydroxypropylsulphonate salts.

21. The composition according to claim 1, wherein the at least one amphoteric surfactant is present in the composition in an amount ranging from 0.1% to 30% by weight, relative to the total weight of the composition.

22. The composition according to claim 21, wherein the at least one amphoteric surfactant is present in the composition in an amount ranging from 1% to 20% by weight, relative to the total weight of the composition.

23. The composition according to claim 22, wherein the at least one amphoteric surfactant is present in the composition in an amount ranging from 1.5% to 15% by weight, relative to the total weight of the composition.

24. The composition according to claim 23, wherein the at least one amphoteric surfactant is present in the composition in an amount ranging from 2% to 8% by weight, relative to the total weight of the composition.

25. The composition according to claim 1, wherein the at least one ceramide compound is present in an amount ranging from 0.0001% to 10% by weight, relative to the total weight of the composition.

26. The composition according to claim 25, wherein the at least one ceramide compound is present in an amount ranging from 0.005% to 5% by weight, relative to the total weight of the composition.

27. The composition according to claim 26, wherein the at least one ceramide compound is present in an amount ranging from 0.01% to 3% by weight, relative to the total weight of the composition.

28. The composition according to claim 1, further comprising at least one additional surfactant chosen from anionic, cationic, nonionic and amphoteric surfactants.

29. The composition according to claim 28, wherein the at least one additional surfactant is present in the composition in an amount ranging from 0.1% to 60% by weight, relative to the total weight of the composition.

30. The composition according to claim 29, wherein the at least one additional surfactant is present in the composition in an amount ranging from 3% to 40% by weight, relative to the total weight of the composition.

31. The composition according to claim 30, wherein the at least one additional surfactant is present in the composition in an amount ranging from 5% to 30% by weight, relative to the total weight of the composition.

32. The composition according to claim 1, further comprising at least one additive chosen from thickeners; fragrances; nacreous agents; opacifiers; preserving agents; sunscreens; cationic surfactants; anionic, nonionic, cationic and amphoteric polymers; proteins; protein hydrolyzates; linear and branched C₁₆-C₄₀ fatty acids; hydroxy acids; vitamins; panthenol; volatile and non-volatile silicones; and fatty esters.

33. The composition according to claim 32, wherein the linear and branched C₁₆-C₄₀ fatty acids are chosen from 18-methyleicosanoic acid.

34. The composition according to claim 32, wherein the cationic polymers are chosen from:
homopolymers of diallyldimethylammonium salt and copolymers of diallyldimethylammonium salt and of acrylamide,
quaternary cellulose ether derivatives,
non-cellulose-based cationic polysaccharides,
polymers comprising repeating units of the formula: 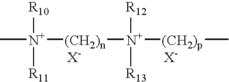
wherein R₁₀, R₁₁, R₁₂ and R₁₃, which may be identical or different, are chosen from C₁-C₄ alkyl and hydroxyalkyl radicals, n and p are integers ranging from 2 to 20, and X⁻ is an anion derived from a mineral or organic acid.

35. The composition according to claim 32, wherein the nacreous agents and/or opacifiers are chosen from titanium oxides; sodium and/or magnesium palmitate; sodium and/or magnesium stearate and hydroxystearate, fatty-chain acyl derivatives, fatty-chain ethers, cyclodextrins, and fatty alcohols.

36. The composition according to claim 35, wherein the fatty-acyl derivatives are chosen from ethylene glycol, polyethylene glycol monostearate, and polyethylene glycol distearate.

37. The composition according to claim 35, wherein the fatty-chain ethers are chosen from C₈-C₃₀ fatty-chain ethers.

38. The composition according to claim 35, wherein the fatty-chain ethers are chosen from distearyl ether and 1-(hexadecyloxy)-2-octadecanol.

39. The composition according to claim 35, wherein the fatty alcohols are chosen from cetyl alcohol, stearyl alcohol and behenyl alcohol.

40. The composition according to claim 1, wherein the composition is in a form chosen from shampoos, conditioners, permanent-waving compositions, relaxing compositions, dyeing compositions, hair bleaching compositions, rinse-out compositions that may be applied between the two steps of a permanent-waving or hair-relaxing operation, and skin washing compositions.

41. A method for washing keratin materials comprising applying to the keratin materials a composition comprising, in a cosmetically acceptable medium, at least one amphoteric surfactant chosen from alkylamphohydroxyalkylsulphonates and salts thereof, and at least one ceramide compound.

42. The method according to claim 41, wherein the keratin materials are hair.

43. A method for treating keratin fibers comprising applying to the keratin materials a composition comprising, in a cosmetically acceptable medium, at least one amphoteric surfactant chosen from alkylamphohydroxyalkylsulphonates and salts thereof, and at least one ceramide compound, and then optionally rinsing with water.

44. A method for improving the fixing of ceramide compounds onto and/or into keratin materials, comprising combining at least one ceramide compound with at least one amphoteric surfactant chosen from alkylamphohydroxyalkylsulphonates and salts thereof.

45. A method of imparting to the hair at least one property chosen from improved disentangling, smoothing, volume, lightness, softness, flexibility, and manageability comprising applying to the hair an effective amount of a composition comprising, in a cosmetically acceptable medium, at least one amphoteric surfactant
chosen from alkylamphohydroxyalkylsulphonates and salts thereof, and at least one ceramide compound.

46. A composition comprising, in a cosmetically acceptable medium, at least one amphoteric surfactant chosen from alkylamphohydroxyalkylsulphonates and salts thereof, and at least one ceramide compound, wherein the least one amphoteric surfactant chosen from alkylamphohydroxyalkylsulphonates and salts thereof, and the at least one ceramide compound, are present in a combined amount effective to impart to the hair at least one property chosen from improved disentangling, smoothing, volume, lightness, softness, flexibility, and manageability.
